(19) United States
(12) Patent Application Publication
(10) Pub. No.: US 2025/0082225 A1
(43) Pub. Date: Mar. 13, 2025

(54) A WRINKLE-MANAGED FILTER SUPPORT SYSTEM ON MOUTHPIECES THAT SHRINK WHEN COOLED DOWN IN THE PRODUCTION PROCESS

(71) Applicant: INOFAB SAGLIK TEKNOLOJILERI ANONIM SIRKETI, Çankaya, Ankara (TR)

(72) Inventors: Merthan Öztürk, Çankaya, Ankara (TR); Kerem Yasar, Çankaya, Ankara (TR); Ahmet Oguz, Çankaya, Ankara (TR); Kadir Tayyip Taslica, Çankaya, Ankara (TR); Ilayda Altun, Çankaya, Ankara (TR); Selim Yonet, Çankaya, Ankara (TR); Çagkan Karaarslan, Çankaya, Ankara (TR); Ceren Yavuz, Çankaya, Ankara (TR)

(73) Assignee: INOFAB SAGLIK TEKNOLOJILERI ANONIM SIRKETI, Çankaya, Ankara (TR)

(21) Appl. No.: 18/728,570

(22) PCT Filed: Dec. 23, 2022
(86) PCT No.: PCT/TR2022/051592

§ 371 Date: Jul. 12, 2024

(30) Foreign Application Priority Data
Jan. 14, 2022 (TR) 2022/000477

(51) Int. Cl. A61B 5/097; A61B 5/00; A61B 5/087; B01D 46/10; B01D 46/56

(52) U.S. Cl. CPC A61B 5/097; A61B 5/087; A61B 5/682; B01D 46/10; B01D 46/56; A61B 2562/12

(57) ABSTRACT

The present invention relates to a method for allowing the filters for protecting against contamination during the passage of the air flow by the sensors in pulmonary function tests to manage the wrinkles on the mouthpieces during production. The object of the present invention is to solve the problems caused by the shrinking of the filter aperture due to the shrinkage of the mouthpiece taken from the mold while cooling after the melting of the plastic raw material of a filter-containing mouthpiece that protects the transceivers from contamination. It is to prevent different mouthpieces from producing different measurement results by ensuring that all mouthpieces produced in succession during serial production show the same wrinkle characteristic in their filters.

### TECHNICAL FIELD

The present invention relates to a method for allowing the filters for protecting against contamination during the passage of the air flow by the sensors in pulmonary function tests to manage the wrinkles on the mouthpieces during production.

### BACKGROUND

One of the methods used during the pulmonary function tests performed with spirometers is the comparison of the flight times of the signals published and read by two reciprocally positioned transceivers, which are angled with an airway through which air flow takes place. Various spirometers operating according to this method are described in documents U.S. Pat. Nos. 5,419,326, 5,647,370, JP2013250254, US2010145213, and WO2019004966. Interchangeable mouthpieces extending along the airway are used to perform respiratory function tests between patients without contamination.

However, the closest patent application in the literature is a patent application numbered 2020/01739 and it is related to the method before this application. In the aforementioned application, the lens effect of wrinkling on the sound wave is mentioned.

During tests performed with spirometers or other pulmonary function test devices, there are windows on the wall of the airway corresponding to transceivers and acting permeably at ultrasonic frequencies so that the signals can interact with the air flow in a healthy way. During the tests, there are filters covering the windows to protect the transceivers from the risk of contamination. The filters have a structure that transmits ultrasonic signals, especially porous. The porous structure is preferably obtained by weaving micrometer-level yarns.

Woven filters can take random wrinkled forms if they are placed on a drawing material before pulling after being taken from the mold due to small changes in production conditions. These filters used on the mouthpieces of spirometers can change the air flow rate, shape, and cross- sectional area during respiratory function tests. As a result of these changes, the path traveled by the signal measuring the air flow rate in the mouthpiece may vary. When the shape/distribution of the filter wrinkle in each mouthpiece is uncontrolled, the measurements made with the mouthpieces produced in series will also differ.

In order to solve this problem, EP3403813A1 describes the tensioning of the filter by thermal processes after the production of the mouthpiece. However, the stretching process creates additional process time and cost.

### THE OBJECT AND BRIEF DESCRIPTION OF THE INVENTION

The object of the present invention is to solve the problems caused by the shrinking of the filter aperture due to the shrinkage of the mouthpiece taken from the mold while cooling after the melting of the plastic raw material of a filter-containing mouthpiece that protects the transceivers from contamination.

The object of the invention is to ensure that wrinkle or corrugated characteristics of all mouthpieces produced in succession during mass production perform low deviating wrinkles without showing great differences. It is to prevent different mouthpieces from producing different measurement results or measurement errors by ensuring that all mouthpieces produced in succession during serial production show the same wrinkle characteristic in their filters. Measurement standards indicate that the measurement values taken from different mouthpieces may contain a maximum of 3% difference from each other or may measure the flowing air volume with a maximum of 50 ml error. Thanks to the support of the filters of the mouthpiece of the invention, it is aimed to double or even triple the sensitivity of these values.

The filter, which is shrunk/crumpled by the shrinking mouthpiece and placed plain at the beginning, will be positioned in the relatively smaller new aperture area by forming two basic forms. These forms are observed as follows:
multiple uncontrolled, scattered peaked wavy wrinkled structures within the aperture,
the bumped-out (b) structure formed by the root mean square of the top and bottom points of the waves.

The invention aims to control both the wavy wrinkled structure and the bump consisting of waves and to give repeatable results in mass production.

### DEFINITIONS OF FIGURES DESCRIBING THE INVENTION

The figures and related descriptions used to better explain the crease-managed filter on the mouthpiece developed by this invention are as follows.

FIG. 1 This is the view of the filter mounted before shrinkage occurs during the initial installation of the filter without the support line during the production of the mouthpiece.

FIG. 2 An AA-Sectional view of the mounted version of the filter before shrinkage occurs during the initial installation of the filter without the support line during the production of the mouthpiece.

FIG. 3 A view of the filter mounted after shrinkage occurs during the first installation of the filter without the support line during the production of the mouthpiece.

FIG. 4 A BB-Sectional view of the mounted version of the filter after shrinkage occurs during the first installation of the filter without the support line during the production of the mouthpiece.

FIG. 5 A view of the filter before the shrinkage of the filter with the zigzag support line during the production of the mouthpiece.

FIG. 6 A CC-Section view of the filter before the shrinkage of the filter with the zigzag support line during the production of the mouthpiece.

FIG. 7 A view of the filter before the shrinkage of the filter occurs together with the curvilinear support line during the production of the mouthpiece.

FIG. 8 A DD-Section view of the filter before the shrinkage of the filter with the curvilinear support line during the production of the mouthpiece.

FIG. 9 A view of the filter after the shrinkage of the filter with the zigzag support line during the production of the mouthpiece.

FIG. 10 An EE-Section view of the filter after the shrinkage of the filter with the zigzag support line during the production of the mouthpiece.

FIG. 11 A view of the filter after the shrinkage of the filter occurs together with the curvilinear support line during the production of the mouthpiece.

FIG. 12 An FF-Section view of the filter after the shrinkage of the filter with the support line during the production of the mouthpiece.

FIG. 13 A perspective view of the entire mouthpiece with zigzag filter supports.

FIG. 14 A perspective view of the entire mouthpiece with filter supports in spring form.

FIG. 15 A perspective view of the entire mouthpiece with filter supports in the form of arrows.

FIG. 16 A perspective view of the state where the filter is not supported on the mouthpiece.

FIG. 17 An alternative GG section of the filter that is taken from the mold since the filter is not supported on the mouthpiece and shows that the filters are bumped out while the cooling mouthpiece is shrinking.

FIG. 18 Another alternative GG section showing that the filters are bumped inward while the cooling mouthpiece is shrinking and taken from the mold since the filter is not supported on the mouthpiece.

FIG. 19 A view of the filter before the shrinkage of the filter occurs together with the arrow-shaped support line during the production of the mouthpiece.

FIG. 20 An HH-Sectional view of the filter before the shrinkage of the filter occurs together with the arrow-shaped support line during the production of the mouthpiece.

FIG. 21 A view of the filter after the shrinkage of the filter with the arrow-shaped support line during the production of the mouthpiece.

FIG. 22 A JJ-Section view of the filter after the shrinkage of the filter with the arrow-shaped support line during the production of the mouthpiece.

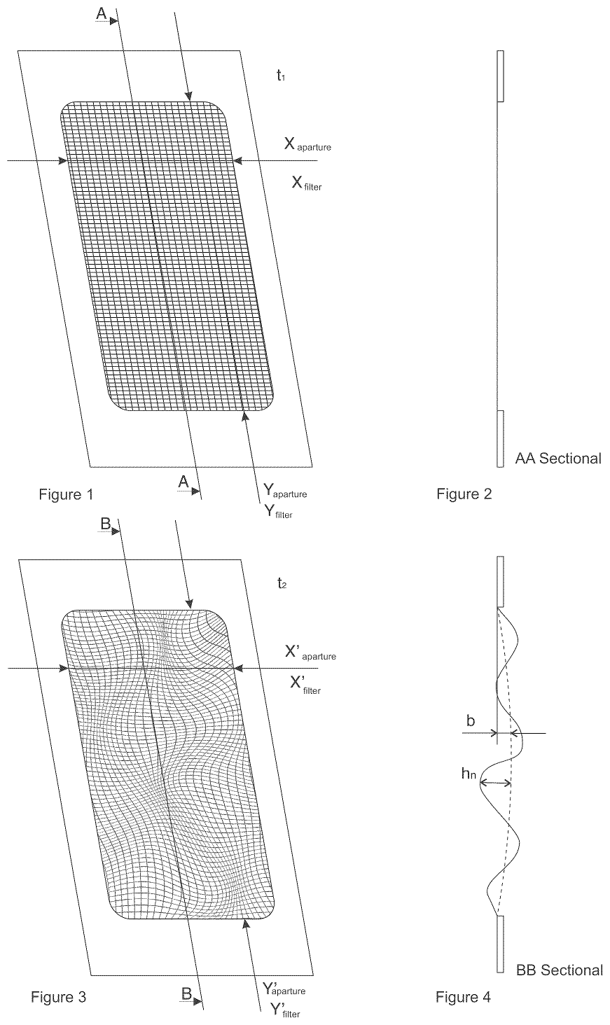

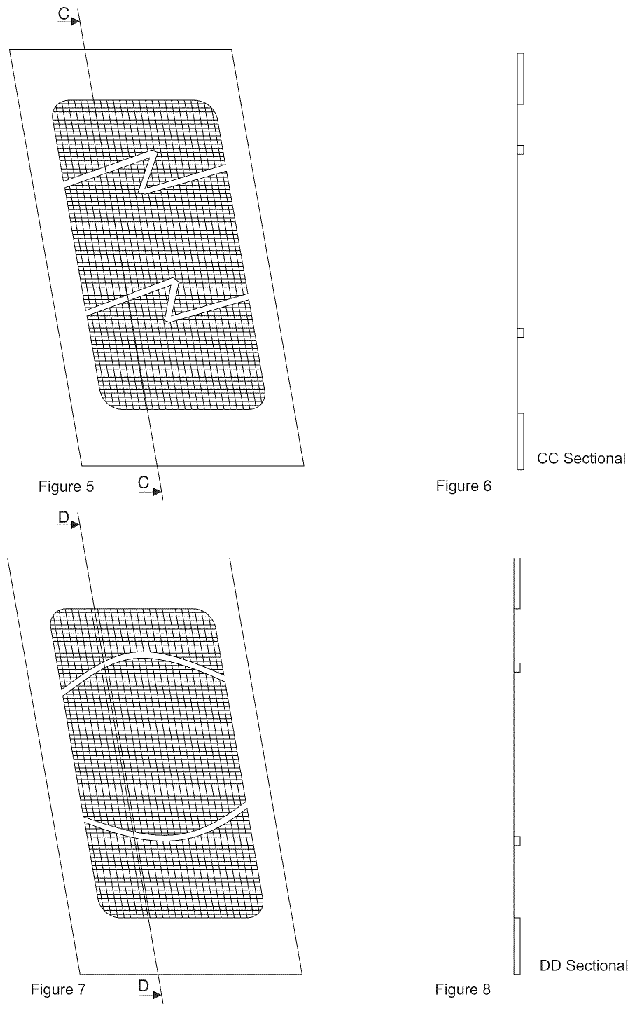

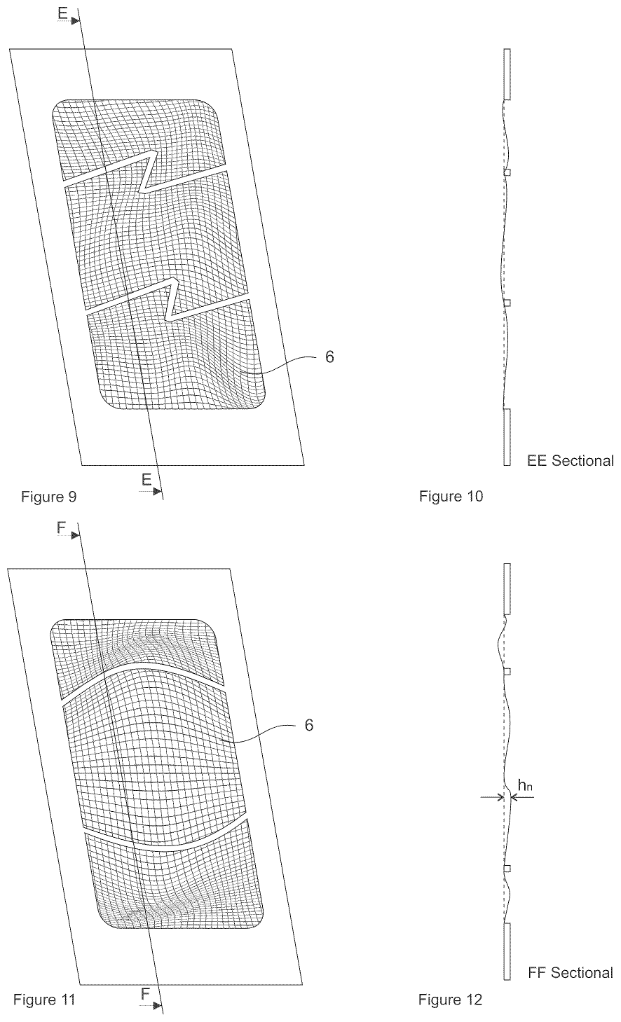

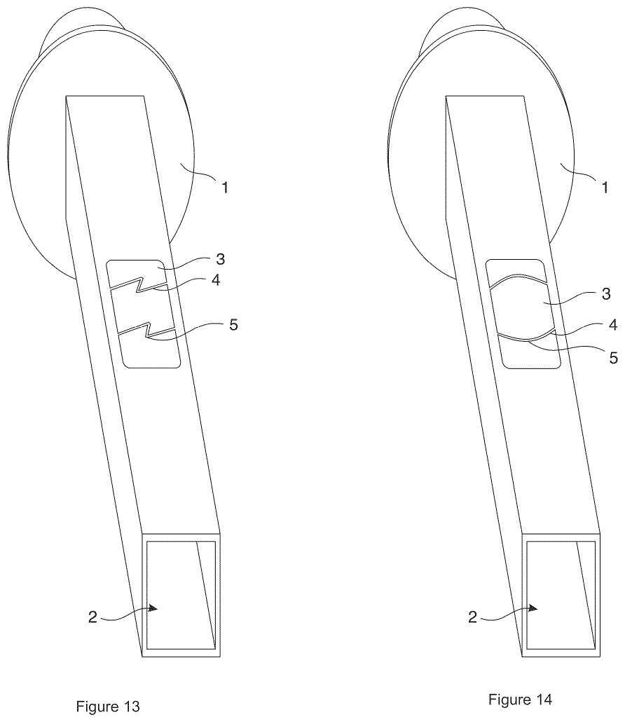

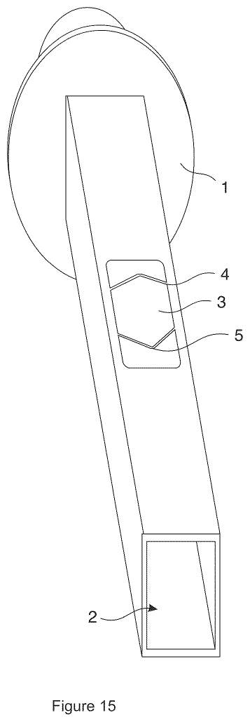

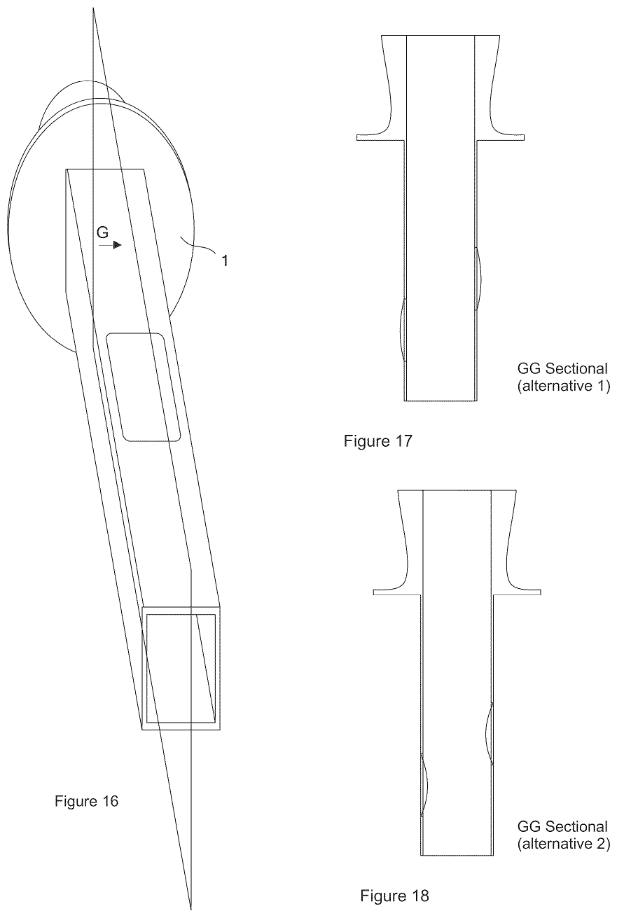

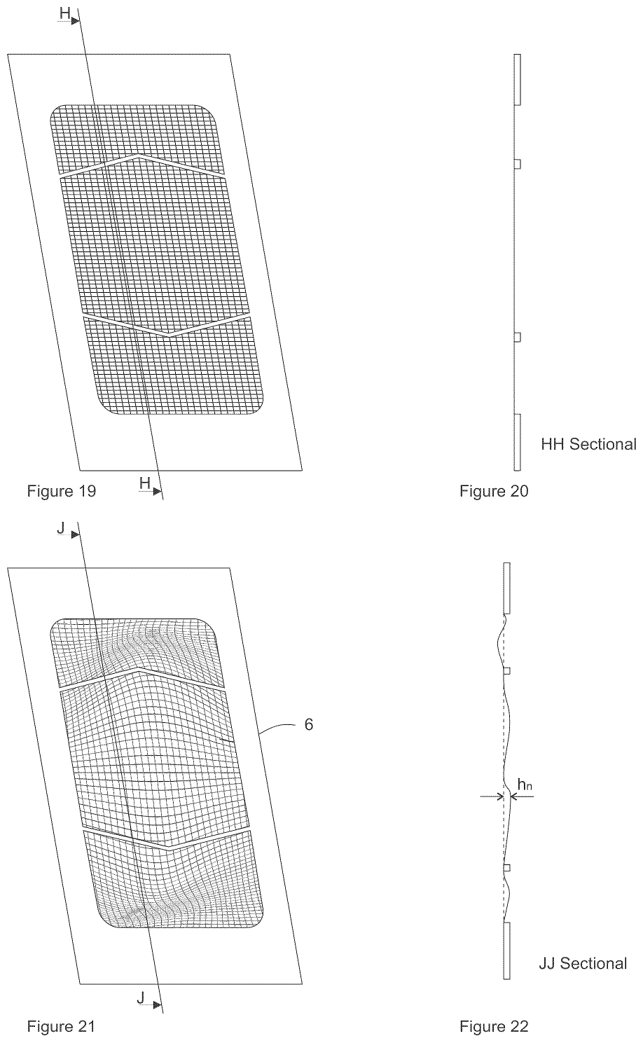

### DEFINITIONS OF ELEMENTS OF THE INVENTION

All elements that provide the wrinkle management developed by this invention are numbered to better explain the subject, and the corresponding of each number is given below.
1. Mouthpiece
2. Airway
3. Filter
4. Support line
5. Puller point
6. Wrinkle region

### DETAILED DESCRIPTION OF THE INVENTION

The mouthpiece (1) of the invention is produced by pressing the plastic raw material in a female-male mold after it is brought to the melting temperature. Due to the production method and the nature of the raw material, the mouthpiece (1) shrinks, that is, pulls from all directions, depending on the temperature, cooling rate, wall thickness, and dimensions when it is taken from the mold while falling to room temperature. Filters (3) are placed on the apertures on the airway (2) of the mouthpiece (1). While the mouthpiece (1) is produced to adhere to the mouthpiece (1), the filters (3) adhere to the apertures on the airway (2) by contacting the edge points because they are larger in width and length.

Since the filters (3) themselves are cold and the melting mouthpiece raw material is hot during the joining, since the filters (3) do not shrink in the same way during the shrinking of the mouthpiece (1), wrinkle regions (6) are formed on the surface of the filter (3). These wrinkle regions (6) are formed in each production regardless of any order due to their chaotic structure. Therefore, the wrinkle region (6) pattern of the filter (3) on each mouthpiece (1) is different from each other.

The velocity of the air passing through the mouthpiece (1) is measured by means of sound waves moving in the space between the two filters (3) in the airway (2). If the wrinkle regions (6) of the filters (3) get closer to each other (forming a concave bump), the cross-sectional area in which the air moves is reduced, and otherwise (forming a convex bump) it grows. Therefore, even if the velocity of the air is the same, different measurement results can be obtained only due to the differences in the wrinkle regions (6).

By making the wrinkle region (6) predictable and reproducible, similar/slightly different wrinkles can be obtained as a result of the mass production of the whole mouthpiece. For this purpose, the filter (3) placed in the aperture will be divided by the support lines (4) produced from the same raw material as the plastic raw material used in the production of the mouthpiece (1) to control the wrinkle regions (6). Support lines (4) are formed by filling plastic raw material into gaps in different geometric shapes on the production molds so that they can be formed on the filters (3) during production.

It is expected that the support lines (4) will cool faster than the mouthpiece (1) body, depending on whether they are thick-thin, or flat-high. It is not technically possible for the support lines (4) to cool more slowly than the body of the mouthpiece (1). Since the support lines (4) showing the same or faster cooling will adhere to the filter (3), they will contribute to the formation of the wrinkle regions (6) and guide the distribution of the wrinkle regions (6) in the aperture. The support lines (4) will move by staying in the plane in a shrinking frame to ensure that they and the filter (3) do not separate from the plane where they were first placed during the shrinking of the aperture.

As explained above, the main cause of wrinkles is that the width (Xₐₚₑᵣₜᵤᵣₑ) and length (Yₐₚₑᵣₜᵤᵣₑ) of the filter decrease during the cooling time (Δt), and the width (X_{filter}) and length (Y_{filter}) of the filter do not change over time despite the new width (X′aperture) and length (Y′ₐₚₑᵣₜᵤᵣₑ).
t₁, Xₐₚₑᵣₜᵤᵣₑ, Yₐₚₑᵣₜᵤᵣₑ
t₂, X′ₐₚₑᵣₜᵤᵣₑ, Yₐₚₑᵣₜᵤᵣₑ,
t₁, X_{filter}, Y_{filter}
t₂, X′_{filter}=X_{filter}, Y′_{filter}=Y_{filter}

Δt=t₂−t₁

The most important object of the invention is to ensure that the placement of the said wrinkles in all wrinkle regions (6) remains the same in each production. The main cause of wrinkling is the X_{filter} and Y_{filter} values that do not change over time (t) despite the change of Xₐₚₑᵣₜᵤᵣₑ−X′ₐₚₑᵣₜᵤᵣₑ and Yₐₚₑᵣₜᵤᵣₑ−Y′ₐₚₑᵣₜᵤᵣₑ values.

With the invention, it is aimed to manage the wrinkling of the filter (3) and especially not to form the bobbin. In order to prevent the wrinkle regions (6) from moving away from the aperture plane, the filter (3) will be actively crumpled in a controlled manner. To achieve this, the shape of the support lines (4) on the filter (3) will be used.

The support lines (4) are positioned to follow different angular and curvilinear paths instead of being positioned parallel to the edges of the filter (3) on the aperture. Thanks to these ways, if the width of the aperture is narrowed, it will pull the filter (3) towards it while on the other hand, it will allow the filter (3) to move away from itself. Two basic forms that enable this function to be performed are described below.
Single-angle arrow or two-angle zigzag form that pushes and pulls the filter (3) with its angular structure

The single- or double-angle zigzag-shaped support line (4) contacts the two long sides of the filter (3) and its length, i.e. the length of the zigzag support line length (X_{support-zigzag}) is greater than the length of the width (X_{filter}) between these two side surfaces.

X_{support-zigzag}>>X_{filter}
Spring form that pushes and pulls the filter (3) with its curvilinear structure

The curvilinear support line (4), which is similar to the single-angle arrow shape, contacts the two long sides of the filter (3), and its length, that is, the length of the curvilinear support line (X_{support-curvilinear}), is greater than the length of the width (X_{filter}) between these two edge surfaces.

X_{support-curvilinear}>>X_{filter}

The length of the curvilinear support line (4) or zigzag support line (4) with a similar single-angle arrow support line (4) is between 1.01 and 1.5 times more than the width (X_{filter}) of the filter (3). A support line (4) of this length contacts the two side walls of the aperture by contacting the filter (3) at 1 or 2 angles/slopes.

In the selection of filter (3) itself, a filter (3) with an open area between 10% and 50% can be used regardless of fiber thickness. In the preferred embodiment of the invention, a filter (3) with an open area between 20-25% will be used.

1. A mouthpiece for preventing the other mouthpieces from producing different measurement results that can be counted as measurement errors by ensuring that plural filters, which protect against contamination during a passage of air flow by sensors in pulmonary function tests, to show common wrinkle characteristics, the mouthpiece comprising:
a body formed from plastic raw material that is pressed in a female-male mold after the plastic raw material is brought to the melting temperature,
two filters for allowing a measurement of a speed of the air passing through an airway in the mouthpiece based on a speed of sound waves moving in space between the two filters,
plural wrinkle regions formed on a surface of at least one of the two filters since shrinkage in the two filters shrink is different from shrinkage in the body as a width (Xₐₚₑᵣₜᵤᵣₑ) and a length (Yₐₚₑᵣₜᵤᵣₑ) at a moment t₁ reach new width (X′ₐₚₑᵣₜᵤᵣₑ) and new length (Y′ₐₚₑᵣₜᵤᵣₑ) values at a moment t₂ after the cooling time of Δt;
plural support lines, wherein each support line has a single angle arrow that pushes and pulls at least one of the two filters using an angular structure of the single angle arrow so that the plural support lines are arranged to actively form a wrinkle remaining in a filter plane on the at least one filter while the aperture is shrinking, and each support line contacts two long edges of the at least one filter and a length support line length (X_{support-arrow}), is between 1.02 and 1.5 times a length of a width (X_{filter}) between the two long edges.

2. A mouthpiece for preventing other mouthpieces from producing different measurement results that can be counted as measurement errors by ensuring that plural filters, which protect against contamination during a passage of air flow by sensors in pulmonary function tests, to show common wrinkle characteristics, the mouthpiece comprising:
a body formed from plastic raw material that is pressed in a female-male mold after the plastic raw material is brought to a melting temperature,
two filters for allowing a measurement of a speed of a air passing through an airway in the body based on a speed of sound waves moving in a space between the two filters,
plural wrinkle regions formed on a surface of the two filters since shrinkage in the two filters is different from shrinkage in the body as a width (Xₐₚₑᵣₜᵤᵣₑ) and length (Yₐₚₑᵣₜᵤᵣₑ) at a moment t₁ reach new width (X′ₐₚₑᵣₜᵤᵣₑ) and new length (Y′ₐₚₑᵣₜᵤᵣₑ) values at a moment t₂ after a cooling time of Δt;
plural support lines, each support line having a biangular zigzag structure that pushes and pulls at least one of the two filters based on the biangular zigzag structure so that the plural support lines are arranged to form a wrinkle that remains in a filter plane on the at least one filter while the aperture is shrinking, and each support line contacts two long edges of the at least one filter and a support line length (X_{support-zigzag}), is between 1.2 and 1.7 times a length of a width (X_{filter}) between the two long edges.

3. A mouthpiece for preventing other mouthpieces from producing different measurement results that can be counted as measurement errors by ensuring that plural filters, which protect against contamination during a passage of air flow by sensors in pulmonary function tests, to show common wrinkle characteristics, the mouthpiece comprising:
a body formed of plastic raw material that is pressed in a female-male mold after the plastic raw material is brought to a melting temperature,
two filters for allowing measurement of a speed of air passing through an airway in the body based on a speed of sound waves moving in a space between the two filters,
plural wrinkle regions formed on a surface of at least one the two filters, wherein shrinkage of the two filters is different from shrinkage of the body as a width (Xₐₚₑᵣₜᵤᵣₑ) and a length (Yₐₚₑᵣₜᵤᵣₑ) at a moment t₁ reach new width (X′ₐₚₑᵣₜᵤᵣₑ) and new length (Y′ₐₚₑᵣₜᵤᵣₑ) values at a moment t₂ after the cooling time of Δt;
plural support lines having a spring that pushes and pulls at least one of the two filters based on an angular structure of the spring so that the plural support lines are arranged to form a wrinkle that remains in a filter plane on the at least one filter while an aperture is shrinking, the plural support lines contact two long edges of the at least one filter and a support line length (X_{support-spring}), is between 1.2 and 1.7 times a length of a width (X_{filter}) between the two long edges.
